(19) United States
(12) Patent Application Publication
(10) Pub. No.: US 2025/0325429 A1
(43) Pub. Date: Oct. 23, 2025

(54) AN INFANT INCUBATOR

(71) Applicant: ARMGATE, SIA, Marupe (LV)

(72) Inventor: Reviks VARDANJANS, Marupe (LV)

(21) Appl. No.: 18/856,614

(22) PCT Filed: Jan. 27, 2023
(86) PCT No.: PCT/LV2023/050002

§ 371 Date: Oct. 13, 2024

(30) Foreign Application Priority Data
May 6, 2022 (LV) LVP2022000039

(51) Int. Cl. A61G 11/00; A61F 7/00; A61G 10/02

(52) U.S. Cl. CPC A61G 11/006; A61F 7/0053; A61G 10/02; A61F 2007/0055

(57) ABSTRACT

The present invention relates to infant incubators with heated walls. The infant incubator (1) comprises a base (2), a mattress (3) arranged on the base (2), a triple walled hood (4) surrounding said base (2) to enclose an infant compartment (5), and a triple walled access door (6) pivotally mounted to the base (2). The triple walled hood (4) and the triple walled access door (6) comprises an inner wall (7), an intermediate wall (8) and an external wall (9) spaced apart from each other so that an inner space (10) and an outer space (11) are formed on each side of the intermediate wall (8). The inner space (10) and the outer space (11) are fluidly connected to each other by means of openings (12) in the intermediate wall (8) so that a warm gas flow (14) is provided within the inner space (10) and the outer space (11).

### FIELD OF THE INVENTION

The present invention relates to infant incubators.

### BACKGROUND OF THE INVENTION

The prior art discloses various infant incubators. The U.S. patent publication No. U.S. Pat. No. 5,100,375 discloses an incubator for infants comprising a hood and a infant compartment enclosed by the hood and wherein air is supplied from an air supply opening arranged between the side walls of the incubator hood and a supporting member defining a supporting surface for accommodating the infant. This incubator is one wall hood incubator with only some parts of the hood to be two walled constructions. Moreover, a pipe is installed into the infant compartment and wherein the pipe is used for collecting the air from an incubator. The U.S patent application publication No. US2015/0065788 discloses an incubator with a double glazed wall. The inner and external glasses are spaced by a spacer, and are characterized by an inner atmosphere confined within the canopy and an external atmosphere.

The U.S. patent publication No. U.S. Pat. No. 4,936,824 discloses an infant incubator which comprises a base, an infant compartment and a hood that encloses the infant compartment. Moreover, the hood is made as the double wall hood allowing a warming air flow re-circulate around the infant compartment. Moreover, the incubator further comprises air curtain. Similar design is disclosed in European patent application publication No. EP0931534.

European patent application publication No. EP0032133 discloses an incubator for infants comprising a base and a partly cylindrical hood enclosing an incubator compartment. Heating of the incubator compartment is achieved by circulating air through a double wall construction of the hood. Similar design is disclosed in the U.S. patent publication No. U.S. Pat. No. 4,750,474.

It is essential to maintain the necessary temperature within the infant incubator. This is not always a case when big temperature difference is present between the inner atmosphere and the external atmosphere. Accordingly, it would be advantageous to design an infant incubator allowing to sustain the necessary temperature within the infant compartment mitigating influence of the external atmosphere.

### SUMMARY OF THE INVENTION

The present invention provides an improved infant incubator having means to allow to sustain the necessary temperature within the inner atmosphere mitigating influence of the external atmosphere.

The aim is reached by a design of an infant incubator comprising a base, a mattress arranged on the base and adapted to support an infant, a hood and an access door. The present invention is characterized in that the hood is a triple walled hood and the access door is a triple walled access door. The triple walled hood surrounding said base to enclose an infant compartment therebetween for containing an infant. The triple walled access door is pivotally mounted to the base.

The triple walled hood and the triple walled access door comprises an inner wall, an intermediate wall and an external wall spaced apart from each other so that an inner space is formed between the inner wall and the intermediate wall and an outer space is formed between the intermediate wall and the external wall. The inner space and the outer space are fluidly connected to each other by means of an opening or openings. The amount and size of an opening and openings are configured based on necessary warm gas flow for necessary incubator volume or infant compartment volume. The inner wall completely seals the infant compartment from the spaces of the triple walled hood and the spaces of the triple walled access door. In result of this an independent air or gas circulation can be provided within the infant compartment and within the triple walled hood and the triple walled access door. This allows to supply an air to the infant compartment specifically suitable for the infant. The air or gas supply in the triple walled hood and in the triple walled access door is specifically adapted for heating purposes in order to maintain a necessary or set temperature within the infant compartment. In other words, the inner wall and the external wall encloses the spaces so that said spaces are completely sealed from the infant compartment and external environment, for example a room in which the incubator is located. This non-existent fluid communication of the spaces with the infant compartment and the external environment allows more energy-effective and agile control or warming of the infant compartment.

The infant incubator comprises a gas flow preparation unit configured to heat and provide a warm gas flow via inlets to the inner space of the triple walled hood and to the inner space of the triple walled access door and further through the openings into the outer space of the triple walled hood and into the outer space of the triple walled access door respectively and further out from said outer spaces via outlets and back to the gas flow preparation unit forming a closed loop of the warm gas flow, in result of which effective heating of the infant compartment is provided. The outer space acts like an insulation to the inner space-less heated, warmed gas flow may be provided maintaining the same characteristics as it would be with double walled incubators. Hence, the present invention is not only more energy efficient but also provides less dangerous (heated) inner wall, which can harm the infant within the incubator.

The gas flow preparation unit comprises a heating means. The heating means may be selected from a group consisting of: gas-conditioned system, an infrared heater, a water/oil heated radiator, a coiled heater, an open coil gas heater, a round open coil gas heater, a convection heater, straight and formed tubular heater, a quartz tube gas heater, a capacitor-type heater, thermoelectric heat-pump. The gas flow is provided by a fan driven by electricity. Any gas flow preparation unit for infant incubators known to the skilled person may be used.

Gas to be warmed and circulated is selected from the group comprising air, xenon, oxygen, nitrogen, carbon dioxide, helium, or a mixture thereof.

The triple walled access door is pivotally mounted to the base by means of a tube hinge which is a part of a tube for providing the warm gas flow from the gas flow preparation unit to the inlets and from the outlets back to the gas flow preparation unit. This tube hinge allows to provide continuous gas flow not only when the door is in closed position but also when the door is in open position. Therefore, there is no need for additional warming up of the door when it has been opened.

The triple walled hood and the triple walled access door are made of transparent material. The wall of the triple walled hood and the triple walled access door may be made of glass, polymers or combinations thereof. For the safety reasons the wall may be laminated so that in the event of breaking it is held in place by said lamination.

In other embodiment of the invention the infant incubator may comprise a triple walled base floor built under the mattress of the incubator. The triple walled base floor comprises an inner wall, an intermediate wall and an external wall spaced apart from each other so that an inner space is formed between the inner wall and the intermediate wall, and an outer space is formed between the intermediate wall and the external wall. The inner space and the outer space are fluidly connected to each other by means of openings. The inner space and the outer space of the triple walled base floor are fluidly connected to the gas flow preparation unit in the same way as it is for the triple walled hood and the triple walled access door.

The inner space of the triple walled hood, the triple walled access door and the triple walled base floor is positioned closer to the infant compartment to be warmed than the outer space of the triple walled hood, the triple walled access door and the triple walled base floor.

The triple walled hood, the triple walled access door and the triple walled base floor may be covered with metallized or electro conductive layer to control heat radiation and to provide localized heating.

The space between the walls of the triple walled hood, the triple walled access door and the triple walled base floor may be filled with or contain desiccant to remove moisture. The desiccant is arranged within the first space and/or the second space so that it does not obstruct the gas flow within the same space.

The gas flow preparation unit provides the warm gas flow to the triple walled hood and separately to the triple walled access door and triple walled base floor so that the infant incubator comprises three separate, independent closed loop warm gas circuits.

### BRIEF DESCRIPTION OF THE DRAWINGS

The drawings illustrate generally, by way of example, but not by way of limitation, various embodiments of the invention.

FIG. 1 is a perspective view of an infant incubator (1) with slightly opened triple walled access doors (6).

FIG. 2 is a side view of the infant incubator (1) as seen in FIG. 1.

FIG. 3 is a cross-section view of a triple walled hood (4), a triple walled access door (6) and a triple walled base floor (20) as seen in A-A of FIG. 2.

FIG. 4 is a cross-section view of a triple walled hood (4) and a triple walled access door (6) as seen in C-C of FIG. 2.

FIG. 5 is a back view of the infant incubator (1) as seen in FIGS. 1 and 2 with principial representation of a gas flow preparation unit (13).

FIG. 6 is a cross-section view of a triple walled hood (4) and a triple walled base floor (20) as seen in B-B of FIG. 5.

FIG. 7. is a perspective view of an infant incubator (1) with removed triple walled hood (4) and triple walled access door (6).

FIG. 8. is a top view of the infant incubator (1) as seen in FIG. 7.

FIG. 9 is a cross-section view of the base (2) of the infant incubator (1) as seen in A-A of FIG. 8.

FIG. 10 is a cross-section view of the base (2) of the infant incubator (1) as seen in B-B of FIG. 8.

FIG. 11 is a cross-section view of the base (2) of the infant incubator (1) as seen in C-C of FIG. 8.

FIG. 12 is a perspective view of another embodiment of the present invention.

### DETAILED DESCRIPTION OF THE EMBODIMENTS

The preferred embodiments of the invention are now described with reference to the figures to illustrate objectives, advantages, and efficiency of the present invention.

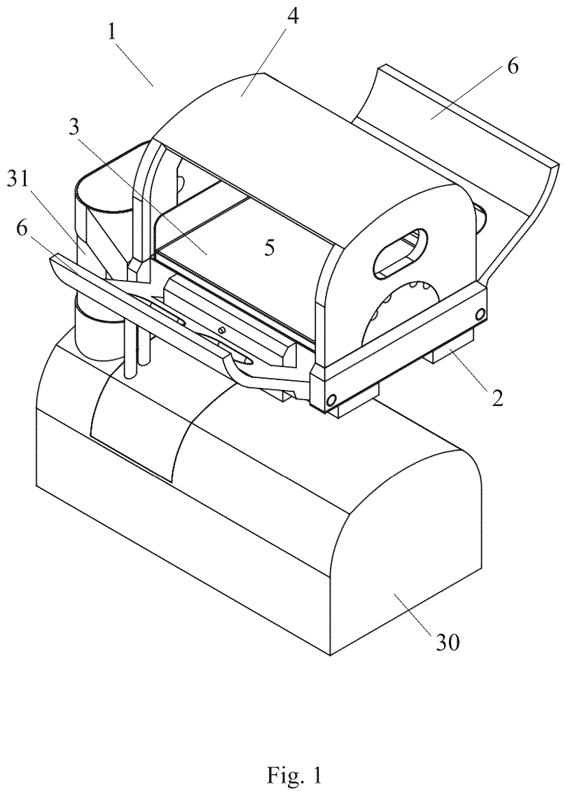

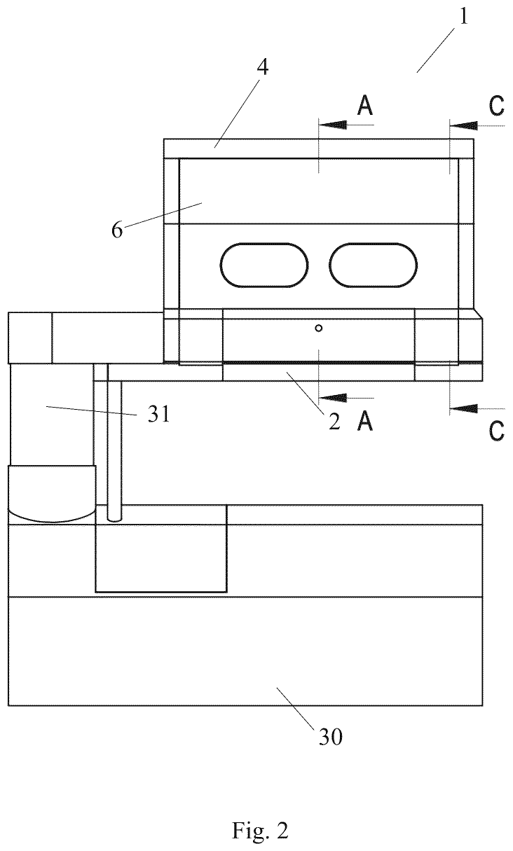

FIG. 1 is a perspective view of an infant incubator (1) and FIG. 2 illustrates the same infant incubator (1) from one of its sides. The infant incubator (1) comprises a base (2). In the present embodiment the base (2) is attached to a column (31) of an incubator carrier (30). The infant incubator (1) comprises a mattress (3) arranged on the base (2) and adapted to support an infant. The infant incubator (1) comprises a triple walled hood (4) surrounding said base (2) to enclose an infant compartment (5) therebetween for containing an infant, and a triple walled access door (6) pivotally mounted to the base (2) further enclosing the infant compartment (5) and at the same time providing access to the infant compartment (5). The triple walled access doors (6) are slightly opened.

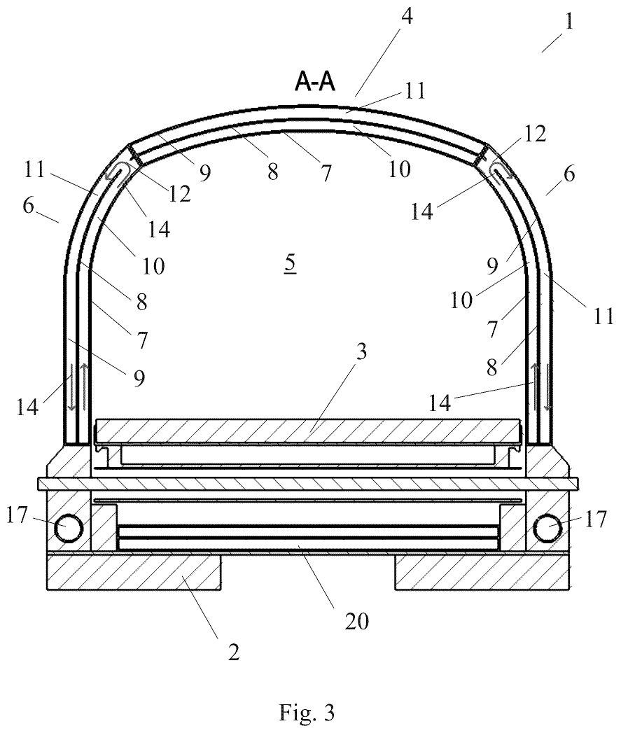

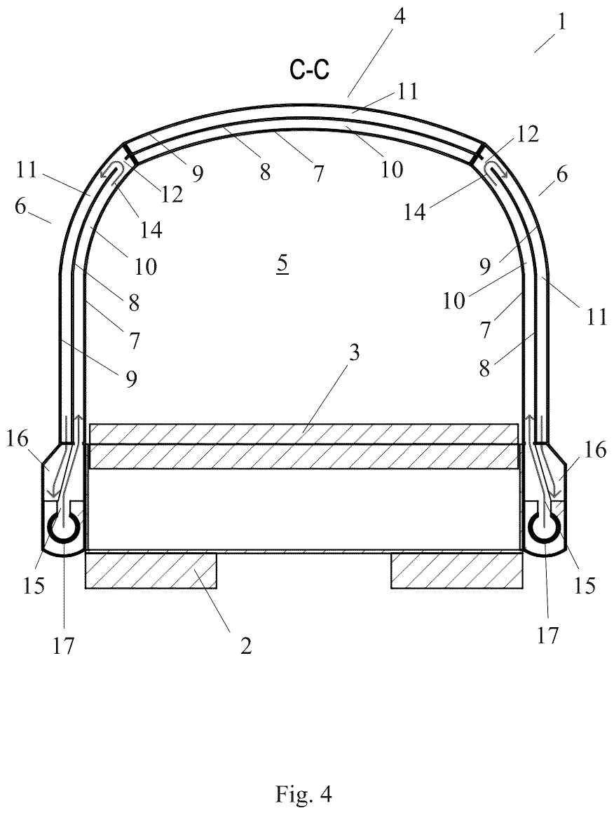

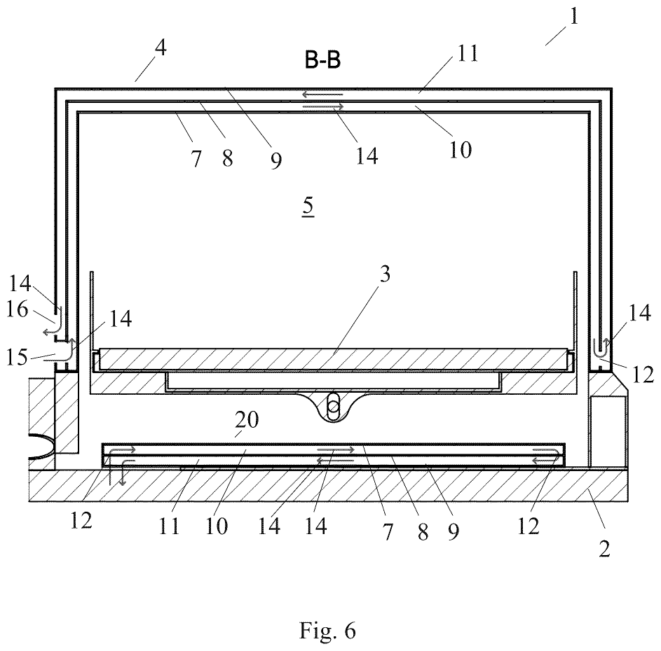

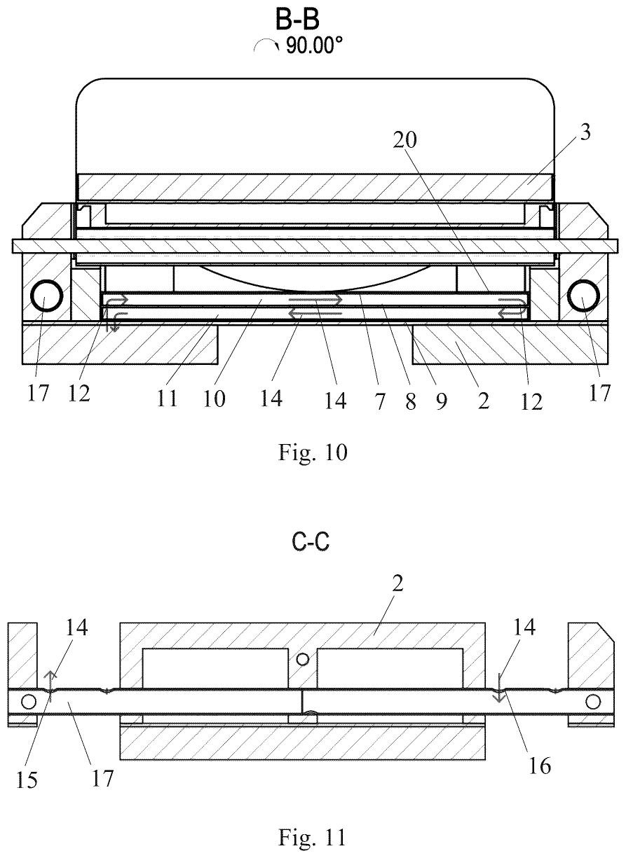

FIG. 3 is a cross-section view of a triple walled hood (4), a triple walled access door (6) and a triple walled base floor (20) as seen in A-A of FIG. 2 and FIG. 4 is a cross-section view of a triple walled hood (4) and a triple walled access door (6) as seen in C-C of FIG. 2 (The triple walled base floor (20) is seen in more detail in FIGS. 6, 9 and 10). The triple walled hood (4) and the triple walled access door (6) comprises an inner wall (7), an intermediate wall (8) and an external wall (9). The inner wall (7) and the intermediate wall (8) are spaced apart from each other so that an inner space (10) is formed therebetween. The intermediate wall (8) and the external wall (9) are spaced apart from each other so that an outer space (11) is formed therebetween. The inner space (10) and the outer space (11) are fluidly connected to each other by means of openings (12). The inner space (10) of the triple walled hood (4) and the triple walled access door (6) is positioned closer to the infant compartment (5) to be warmed than the outer space (11) of the triple walled hood (4) and the triple walled access door (6).

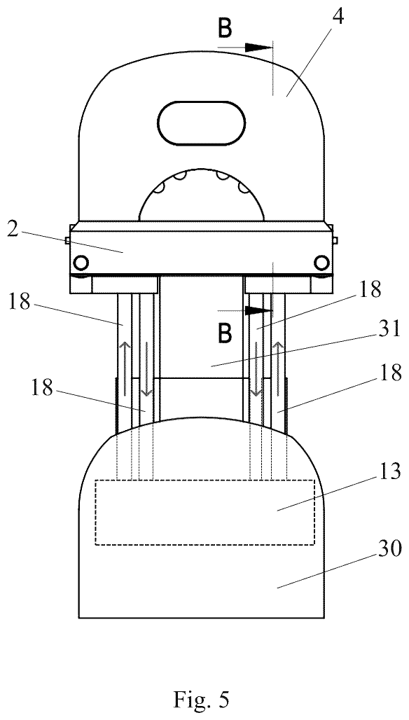

The infant incubator comprises a gas flow preparation unit (13) configured to heat and provide a warm gas flow (14) to the triple walled hood (4), the triple walled access door (6) and a triple walled base floor (20) (see FIG. 5). The gas flow preparation unit (13) is installed in the carrier (30) of the infant incubator (1) and the gas flow preparation unit (13) is connected to the triple walled hood (4), the triple walled access door (6) and a triple walled base floor (20) through conduits (18). The gas flow preparation unit comprises a heating means for heating the gas flow and a fan for providing said gas flow. The warm gas flow (14) is provided to the inner space (10) of the triple walled hood (4) and to the inner space (10) of the triple walled access door (6) via inlets (15) as seen in FIGS. 6 and 11. The warm gas flow (14) is further conducted through the openings (12) in the intermediate wall (8) into the outer space (11) of the triple walled hood (4) and the outer space (11) of the triple walled access door (6). The warm gas flow (14) is further conducted out from said outer spaces (11) via outlets (16) and back to the gas flow preparation unit (13) forming a closed loop of the warm gas flow (14) as seen in FIGS. 2, 4, 5 and 6. The warm gas flow (14) does not enter or exit the infant compartment (5), as well as does not enter or exit to an external environment. Hence, effective heating of the infant compartment (5) is provided.

The triple walled base floor (20) is seen in more detail in FIGS. 6, 9 and 10. The triple walled base floor (20) comprises an inner wall (7), an intermediate wall (8) and an external wall (9) spaced apart from each other. An inner space (10) is formed between the inner wall (7) and the intermediate wall (8). An outer space (11) is formed between the intermediate wall (8) and the external wall (9). The inner space (10) and the outer space (11) are fluidly connected to each other by means of openings (12) and said inner space (10) and outer space (11) of the triple walled base floor (20) are fluidly connected to the gas flow preparation unit (13).

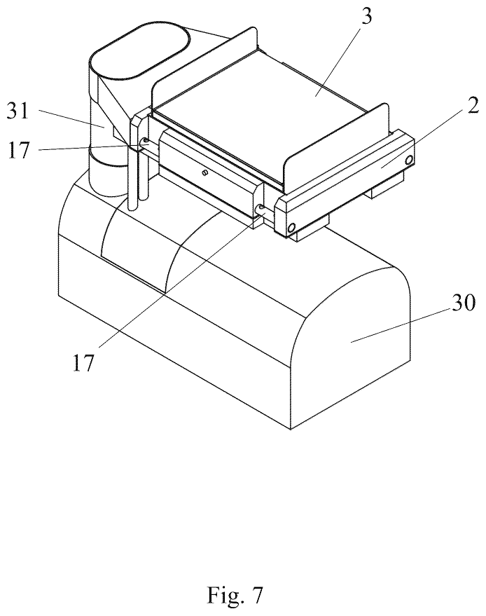

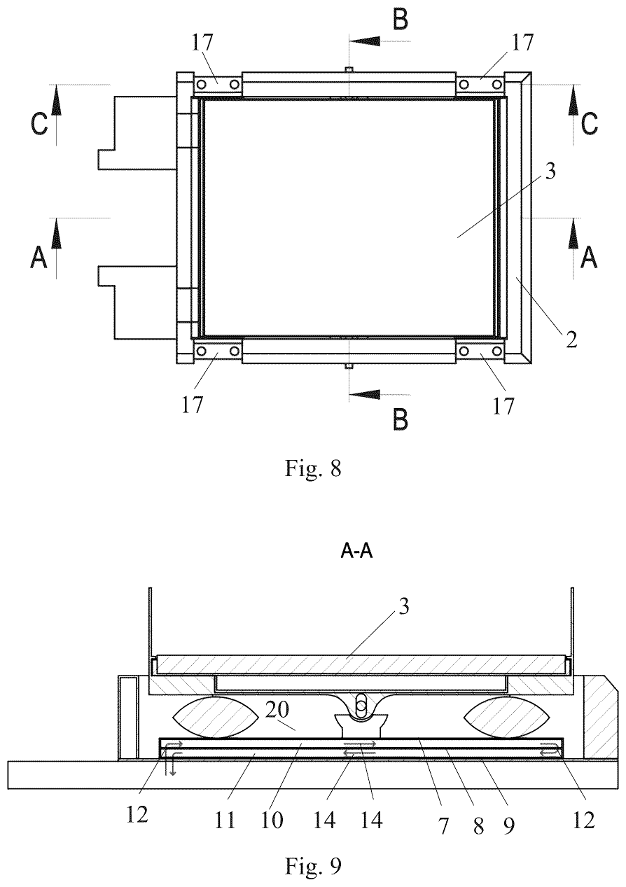

The triple walled access door (6) is pivotally mounted to the base (2) by means of a tube hinge (17) which is a part of a tube for providing the warm gas flow (14) from the gas flow preparation unit (13) to the inlets (15) and from the outlets (16) back to the gas flow preparation unit (13). See FIGS. 3, 4, 7, 8, 10 and 11.

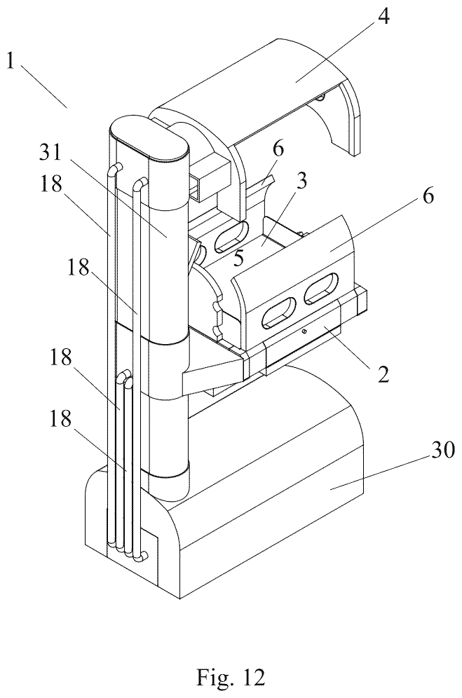

FIG. 12 is a perspective view of another embodiment of the present invention. The infant incubator (1) comprises extendable column (31) compared to the previously described embodiment allowing to lift and lower the triple walled hood (4) vertically. This design allows to remove the triple walled hood (4) and triple walled access doors (6) so that there is full, non-restricted access to the infant compartment (5).

While the invention may be susceptible to various modifications and alternative forms, specific embodiments of which have been shown by way of example in the figures and have been described in detail herein, it should be understood that the invention is not intended to be limited to the particular forms disclosed. Rather, the invention includes all modifications, equivalents, and alternatives falling within the scope of the invention as defined by the following claims.

1. An infant incubator (1) comprising:
a base (2),
a mattress (3) arranged on the base (2) and adapted to support an infant,
a triple walled hood (4) surrounding said base (2) to enclose an infant compartment (5) therebetween for containing an infant,
a triple walled access door (6) pivotally mounted to the base (2),
the triple walled hood (4) and the triple walled access door (6) comprises an inner wall (7), an intermediate wall (8) and an external wall (9) spaced apart from each other so that an inner space (10) is formed between the inner wall (7) and the intermediate wall (8), and an outer space (11) is formed between the intermediate wall (8) and the external wall (9), wherein the inner space (10) and the outer space (11) are fluidly connected to each other by means of openings (12) in the intermediate wall (8), wherein the inner space (10) of the triple walled hood (4) and the triple walled access door (6) is positioned closer to the infant compartment (5) to be warmed than the outer space (11) of the triple walled hood (4) and the triple walled access door (6), and wherein the inner wall (7) and the external wall (9) encloses the spaces (10, 11) so that said spaces (10, 11) are completely sealed from the infant compartment (5) and external environment,
a gas flow preparation unit (13) configured to heat and provide a warm gas flow (14) via inlets (15) to the inner space (10) of the triple walled hood (4) and to the inner space (10) of the triple walled access door (6) and further through the openings (12) into the outer space (11) of the triple walled hood (4) and the outer space (11) of the triple walled access door (6) respectively and further out from said outer spaces (11) via outlets (16) and back to the gas flow preparation unit (13) forming a closed loop of the warm gas flow (14), in result of which effective heating of the infant compartment (5) is provided.

2. The infant incubator (1) according to claim 1, characterized in that the triple walled access door (6) is pivotally mounted to the base (2) by means of a tube hinge (17) which is a part of a tube for providing the warm gas flow (14) from the gas flow preparation unit (13) to the inlets (15) and from the outlets (16) back to the gas flow preparation unit (13).

3. The infant incubator (1) according to claim 1, characterized in that the base (2) comprises a triple walled base floor (20) arranged below the mattress (3), wherein the triple walled base floor (20) comprises an inner wall (7), an intermediate wall (8) and an external wall (9) spaced apart from each other so that an inner space (10) is formed between the inner wall (7) and the intermediate wall (8), and an outer space (11) is formed between the intermediate wall (8) and the external wall (9), wherein the inner space (10) and the outer space (11) are fluidly connected to each other by means of openings (12) and said inner space (10) and outer space (11) of the triple walled base floor (20) are fluidly connected to the gas flow preparation unit (13).

4. The infant incubator (1) according to claim 1, characterized in that the triple walled hood (4) and the triple walled access door (6) are made of transparent material.

5. The infant incubator (1) according to claim 1, characterized in that the triple walled hood (4), the triple walled access door (6) and the triple walled base floor (20) is covered with metallized or electroconductive layer.

6. The infant incubator (1) according to claim 1, characterized in that the gas flow preparation unit (13) provides the warm gas flow (14) to the triple walled hood (4) and separately to the triple walled access door (6) and triple walled base floor (20) so that the infant incubator (1) comprises three separate closed loop warm gas circuits.
